(19) United States
(12) Patent Application Publication
(10) Pub. No.: US 2014/0111768 A1
(43) Pub. Date: Apr. 24, 2014

(54) OPHTHALMOLOGIC APPARATUS, OPHTHALMOLOGICAL CONTROL METHOD, AND STORAGE MEDIUM

(71) Applicant: CANON KABUSHIKI KAISHA, Tokyo (JP)

(72) Inventor: Isao Komine, Sagamihara-shi (JP)

(73) Assignee: CANON KABUSHIKI KAISHA, Tokyo (JP)

(21) Appl. No.: 14/061,321

(22) Filed: Oct. 23, 2013

(30) Foreign Application Priority Data
Oct. 24, 2012 (JP) 2012-234649

(51) Int. Cl. A61B 3/14

(52) U.S. Cl. CPC A61B 3/14
USPC 351/206; 351/246

(57) ABSTRACT

The ophthalmologic apparatus includes a light source configured to irradiate a subject's eye, an irradiation optical system arranged between the light source and the subject's eye, a first image forming optical system configured to include a first optical path splitting member for splitting an optical path of the irradiation optical system and form an image of a return light from the subject's eye as a first image, and a second image forming optical system configured to include a second optical path splitting member for splitting an optical path of the irradiation optical system and form an image of a light output from the light source without through the subject's eye as a second image on a different position from that of the first image.

### BACKGROUND OF THE INVENTION

1. Field of the Invention

The present invention relates to an ophthalmologic apparatus, an ophthalmological control method, and a storage medium.

2. Description of the Related Art

Currently, various standards for medical devices have been introduced, and with respect to ophthalmologic devices for performing inspection, measurement, processing, and so on, realization of an apparatus using a light amount which is safe for a subject's eye is required. At the same time, improvement in performance of an apparatus is also essential for further accurate diagnoses corresponding to various subjects. Therefore, a laser light source with a high light amount or the like needs to be used. Thus, development of an excellent interlocking mechanism is required which can secure the safety of the subjects.

As a conventional technique, an optical coherence tomography (OCT) apparatus is known which opens and closes a shutter or the like for blocking an optical path based on a light amount of a reference light (measurement is performed if the light amount of the reference light is in an allowable range and not performed if the light amount of the reference light is out of the allowable range). (See Japanese Patent Application Laid-Open No. 2011-27715.)

However, the technique discussed in Japanese Patent Application Laid-Open No. 2011-27715 switches whether to irradiate a subject's eye with a measurement light based on a light amount of a reference light which is detected in a state that the subject's eye is not irradiated with the measurement light. Based on this technique, detection of the light amount of the reference light in a state that the subject's eye is irradiated with the measurement light is not performed, thus there is a room for improvement so as not to apply an inappropriate laser beam to the subject's eye during the irradiation.

### SUMMARY OF THE INVENTION

The present invention is directed to an ophthalmologic apparatus capable of recognizing whether an inappropriate amount of light (an excessive amount of light) is not used in irradiation in a case where a subject's eye is irradiated with a measurement light, and an the ophthalmological control method and a storage medium therefore.

According to an aspect of the present invention, an ophthalmologic apparatus includes a light source configured to irradiate a subject's eye, an irradiation optical system arranged between the light source and the subject's eye, a first image forming optical system configured to include a first optical path splitting member for splitting an optical path of the irradiation optical system and form an image of a return light from the subject's eye as a first image, and a second image forming optical system configured to include a second optical path splitting member for splitting an optical path of the irradiation optical system and form an image of a light output from the light source without through the subject's eye as a second image on a different position from that of the first image

According to another aspect of the present invention, a method for ophthalmological control includes irradiating a subject's eye by an irradiation optical system arranged between a light source and the subject's eye, forming an image of the light source reflected by the subject's eye as a first image by a first image forming optical system including a first optical path splitting member for splitting an optical path of the irradiation optical system, and forming an image of the light source without through the subject's eye as a second image on a different position from that of the first image by a second image forming optical system including a second optical path splitting member for splitting an optical path of the irradiation optical system

According to yet another aspect of the present invention, there is provided an ophthalmological control program.

Further features of the present invention will become apparent from the following description of exemplary embodiments with reference to the attached drawings.

### BRIEF DESCRIPTION OF THE DRAWINGS

FIG. 1A illustrates an example of an optical configuration of an eye refractive power measurement apparatus according to a first exemplary embodiment of the present invention, and FIG. 1B is a functional block diagram thereof.

FIG. 2 illustrates an example of an arrangement of a measurement ring image and a reference spot image on an image sensor according to the first exemplary embodiment.

FIG. 3A is a flowchart illustrating an example of operations according to the first exemplary embodiment. FIG. 3B is a flowchart illustrating detail processing in step S4. FIG. 3C illustrates a number of signals which are detected on each scanning line position.

FIG. 4 illustrates an example of an optical configuration of an eye refractive power measurement apparatus according to a modification.

FIG. 5 is a perspective view of an alignment prism diaphragm according to an exemplary embodiment of the present invention.

FIG. 6A illustrates a state in which an alignment in a back-and-forth direction is adjusted using an alignment prism diaphragm. FIG. 6B illustrates a state in which the alignment is too far. FIG. 6C illustrates a state in which the alignment is too close.

### DESCRIPTION OF THE EMBODIMENTS

Various exemplary embodiments, features, and aspects of the invention will be described in detail below with reference to the drawings.

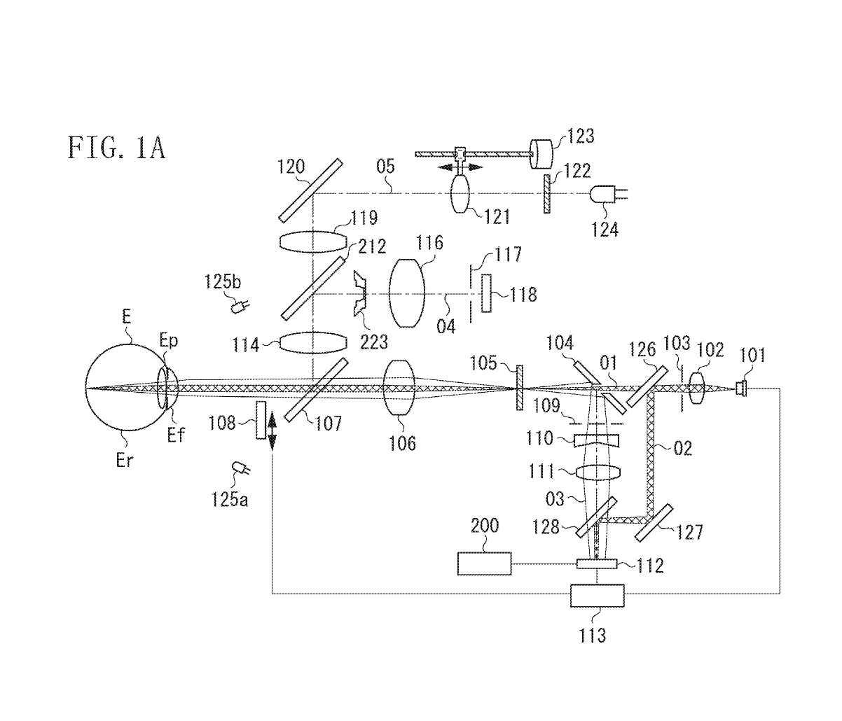

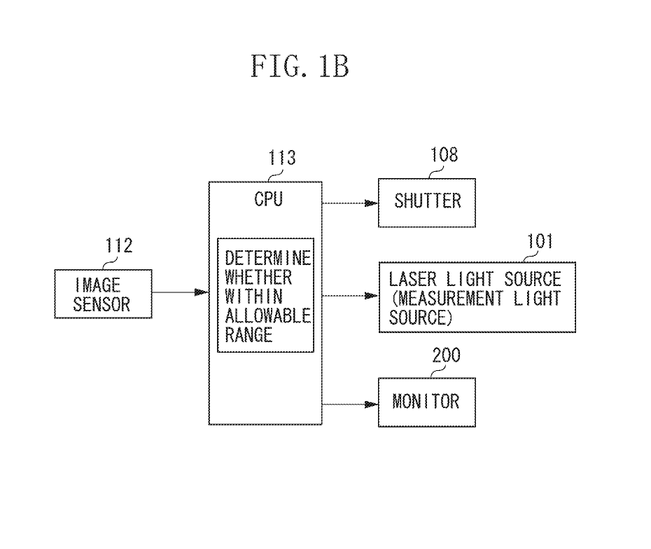

FIG. 1A illustrates an example of an optical configuration of an eye refractive power measurement apparatus according to a first exemplary embodiment of the present invention.

### (Fixation Target Projection Optical System and Alignment Light-Receiving Optical System)

First, in a reflection direction of a dichroic mirror 107, a fixation target projection optical system and an alignment light-receiving optical system which is used for both of an anterior eye portion observation and an alignment detection of a subject's eye are arranged. On an optical path 05 in the fixation target projection optical system, a lens 114, a dichroic mirror 212, a lens 119, a folding mirror 120, a lens 121, a fixation target 122, and a fixation target illumination light source 124 are arranged in this order.

At the time of guiding a fixation, a projected light beam of the lit fixation target illumination light source 124 illuminates the fixation target 122 from a backside thereof, and is projected onto a fundus Er of a subject's eye E via the lens 121, the folding mirror 120, the lens 119, and the lens 114. In addition, the lens 121 can be moved in an optical axis direction by a fixation guiding motor 123 for guiding a diopter of the subject's eye E to achieve a fogging state thereof.

On an optical path 04 in the reflection direction of the dichroic mirror 212, an alignment prism diaphragm 223, a lens 116, a diaphragm 117, and an image sensor 118 are arranged in this order, thus the anterior eye portion observation and the alignment detection of the subject's eye can be performed. The alignment prism diaphragm 223 is driven by an alignment prism diaphragm drive solenoid (not illustrated), and the diaphragm 117 is driven by a diaphragm drive solenoid (not illustrated).

By insertion and retraction of the alignment prism diaphragm 223, when the alignment prism diaphragm 223 is on the optical path 06, the alignment can be performed, and when the alignment prism diaphragm 223 is retracted from the optical path 06, the anterior eye portion observation or the transillumination observation can be performed.

The alignment prism diaphragm 223 includes three openings (a center opening 223a, and openings 223b and 223c on both sides in a lateral direction) on a disk-shaped diaphragm plate as illustrated in FIG. 5. On the dichroic mirror 212 side of the openings 223b and 223c on both sides in the lateral direction, alignment prisms 301a and 301b for transmitting only light beams of wavelength of, for example, near 880 nm are respectively attached.

In addition, anterior eye portion illumination light sources 125a and 125b of wavelength, for example, about 780 nm are arranged diagonally forward of an anterior eye portion of the subject's eye E. A light beam from the anterior eye portion of the subject's eye which is illuminated by the anterior eye portion illumination light sources 125a and 125b passes through the dichroic mirror 107, the lens 114, the dichroic mirror 212, and the center opening 223a of the alignment prism diaphragm 223, and is focused on a light receiving sensor surface of an image sensor 118. Here, the center opening 223a of the alignment prism diaphragm 223 transmits a light beam of wavelength equal to or greater than 780 nm from anterior eye portion illumination light sources 125a and 125b.

### (Alignment)

A measurement light source 101 is used as both a light source for alignment detection and a light source for eye refractive power measurement. At the time of alignment, a semi-transparent diffusion plate 105 is inserted into the optical path by a diffusion plate drive solenoid. A position where the diffusion plate 105 is inserted is a primary focusing position of a projection lens 102 of the measurement light source 101, and further the diffusion plate 105 is inserted to a focal position of a lens 106. Accordingly, an image of the measurement light source 101 is once focused on the diffusion plate 105, and then projected from the lens 106 toward the subject's eye E as a secondary light source with a thick parallel light beam.

This parallel light beam is reflected by a cornea Ef of the subject's eye and forms a bright spot image. Then, the light beam is partially reflected again by the dichroic mirror 107, and further reflected by the dichroic mirror 212 via the lens 114. Further, the light beam passes through the alignment prism diaphragm 223, is converged by the lens 116, and focused on an image sensor 118

In other words, the light beam divided by the openings 223a, 223b, and 223c of the alignment prism diaphragm 223 and the prisms 301a and 301b are formed as index images Ta, Tb, and Tc on the image sensor 118. In addition, bright spot images of external eye illumination light sources are captured by the image sensor 118 together with images of the anterior eye portion of the subject's eye illuminated by the external eye illumination light sources.

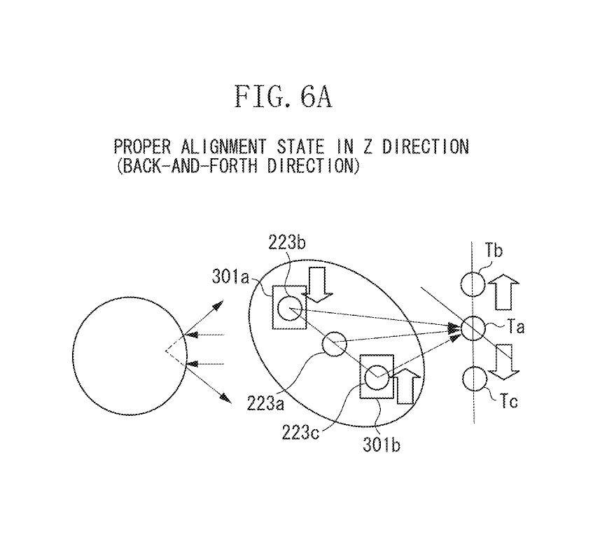

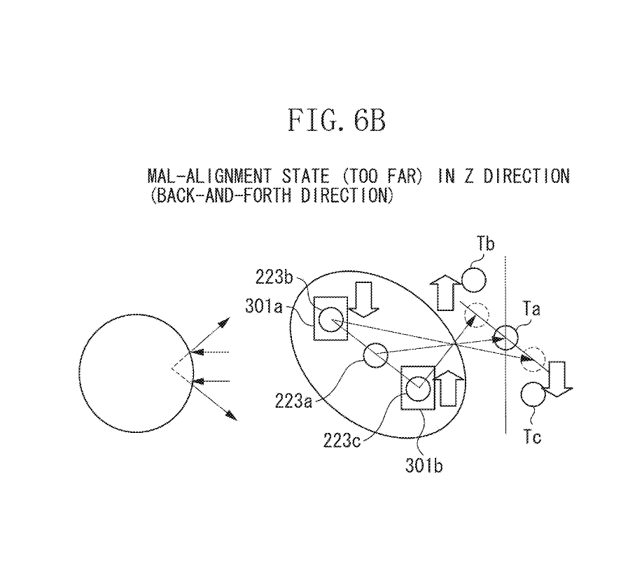

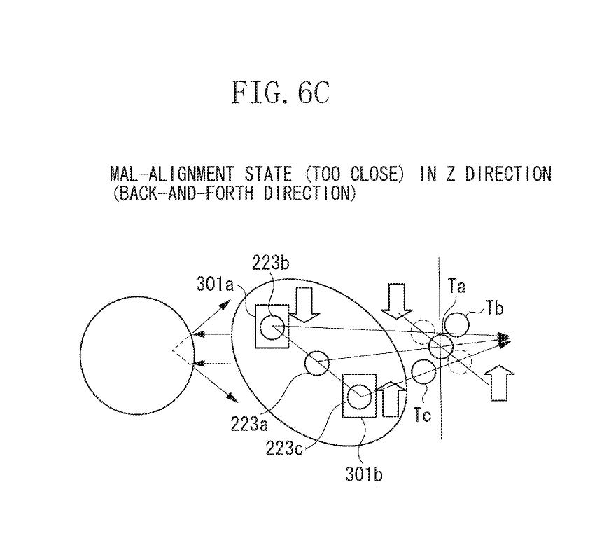

As illustrated in FIG. 6A, alignment is completed in a state in which three cornea bright spots Ta, Tb, and Tc are aligned in a direction perpendicular to the horizontal direction. When the alignment is in a poor state in a Z direction (i.e., the back-and-forth direction), the alignment is to be a state as illustrated in FIG. 6B when it is too far, and to be a state as illustrated in FIG. 6C when it is too close.

### (Refractive Power Measurement)

An optical system including the optical path 01 is used for eye refractive power measurement. A light beam output from the measurement light source 101 is narrowed by a diaphragm 103, primarily focused on short of the lens 106 by a lens 102, and then projected onto a center of a pupil of the subject's eye E after passing through the lens 106 and the dichroic mirror 107. The light beam is focused on the fundus Er, and the reflected light thereof is incident on the lens 106 again after passing through the center of the pupil. The incident light beam is reflected on the periphery of a perforated mirror 104 after passing through the lens 106. The perforated mirror 104 functions as a first optical path splitting member for splitting the optical path 01 of an irradiation optical system which irradiates the subject's eye.

The reflected light beam is subjected to pupil separation by a ring-shaped diaphragm 109 which is conjugate to the pupil Ep of the subject's eye in a first image forming optical system including the lenses 106 and 111, and is formed as a ring image on a light-receiving surface of an image sensor 112 via a prism 110 for displacing the light beam to each meridian direction. If the subject's eye E is an emmetropia, the ring image will be a predetermined circle. In the case of a myopia, a curvature of the circle will be smaller, and in the case of hyperopia, a curvature of the circle will be larger. The subject's eye E is an astigmatic eye, the ring image will be an ellipse, and an angle between a horizontal axis and a major axis of the ellipse is an astigmatic axis angle. A refractive power is calculated based on a coefficient of the ellipse. An examiner can visually check an image captured by the image sensor 112 by a monitor 200.

### (Reference Light for Determination of Laser Irradiation)

According to the present exemplary embodiment, a super luminescent diode (SLD) for generating a laser beam with a wavelength of 880 nm, which is a near-infrared light, is used as an example of the measurement light source 101 for eye refractive power measurement. The measurement light source 101 can be also used as a light source of a reference light for determination of laser irradiation, which is described below.

An optical path 02 through which a light reflected by a beam splitting mirror 126 passes is an optical path for monitoring a part of a projected light projected by the laser light source 101 as the reference light via the optical path 02. The beam splitting mirror 126 functions as a second optical path splitting member for splitting the optical path 01 of the irradiation optical system for irradiating the subject's eye. In other words, the laser beam emitted from the measurement light source 101 is formed as a spot image on an image sensor in a second image forming optical system including the lens 102, the diaphragm 103, the beam splitting mirror 126, a mirror 127, and a beam splitting mirror 128.

On the other hand, after being reflected by the subject's eye E, the measurement light is subjected to pupil separation by the ring-shaped diaphragm 109, and formed as a ring image on the light-receiving surface of the image sensor 112 via the prism 110 and a lens 111. In order to make the measurement light into a ring-shaped light beam and make the reference light into a spot-shaped light beam, a position at which the reference light enters the optical path 03 (namely, a position of the beam splitting mirror 128) needs to be closer to the image sensor 112 than the diaphragm 109.

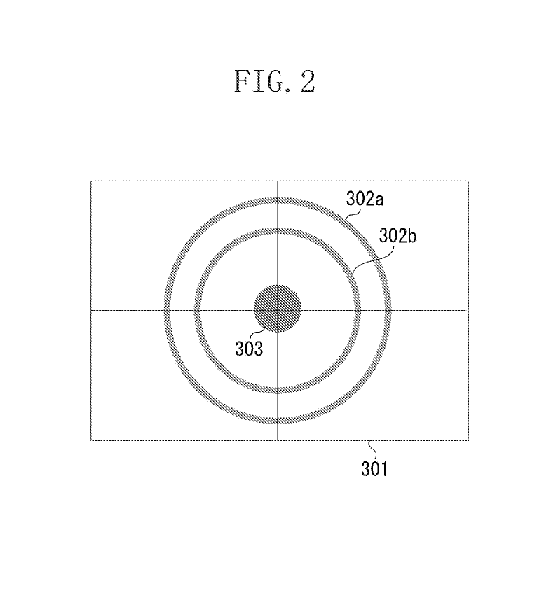

FIG. 2 illustrates a positional relationship between a ring image of the measurement light and a spot image of the reference light formed within the ring image on the image sensor 112 according to the present exemplary embodiment. The image sensor 112 has an effective pixel surface 301. A ring image 302a is an image formed when a diopter of a subject's eye is at the most plus side, and a ring image 302b is an image formed when a diopter of a subject's eye is at the most minus side. Therefore, an area closer to the center than the ring image 302b is not used in the measurement. A spot image 303 is formed by the reference light and positioned around the center so as not to overlap with the ring image 302b.

On a two-dimensional surface of the image sensor 112, it is set in which area the most plus side ring image and the most minus side ring image regarding the diopter are respectively captured within a measurable range of the eye refractive power. In other words, an area on the two-dimensional surface of the image sensor 112 which is not used in the detection of the ring image is known in advance. The spot image of the reference light is formed on the area, and thus a state of the laser light source can be monitored by the reference light during the measurement of the ring image. In this regard, the positional relationship between the ring image and the spot image is not limited to the one illustrated in FIG. 2. A positional relationship other than the one in FIG. 2 can be taken as long as a ring image and a spot image do not overlap with each other in their positional relationship. (Difference in Receiving Light Amount between Measurement Light

and Reference Light)

The measurement light and the reference light are light beams which are emitted from the same laser light source, however, respective optical paths to the image sensor 112 are different, so that respective receiving light amounts on the image sensor are different. In order to measure the measurement light and the reference light at the same time, it is desirable to set the same measurement gain in the image sensor, and to reduce a difference in the receiving light amount between the measurement light and the reference light. Setting the same measurement gain enables the measurement of the reference light and the measurement light without gain adjustment at the measurement. In addition, since a noise level is the same, measurement errors of the reference light and the measurement light can be reduced.

If terms “simultaneous”, “same”, and the like are used in the description of the present invention, they include concepts not only a case which means exactly the same time and identical but also a case which means approximately the same time and approximately identical.

In FIG. 1A, an attenuation rate of the measurement light is determined on the way from the dichroic mirror 107 to the beam splitting mirror 128 on the optical path 03 via the perforated mirror 104. In the present exemplary embodiment, it is assumed that a transmittance of the dichroic mirror 107 is 90%, a reflectance when the incident light is reflected by the fundus of the subject's eye and returned is 0.1%, the transmittance when the light beam again passes through the dichroic mirror 107 is 90%, and there is no loss by the reflection by the perforated mirror 104. In addition, a transmittance of the prism 110 in the optical path 03 after being reflected by the perforated mirror 104 is 90%, the attenuation rate of the measurement light will be approximately 0.15% .

On the other hand, an attenuation rate of the reference light is determined on the way from the beam splitting mirror 126 to the beam splitting mirror 128 on the optical path 02. Since the attenuation rate of the measurement light is approximately 0.15%, if the attenuation rate 0.15% is compensated only by the beam splitting mirror 126, the transmittance to the optical path 01 may be set to about 99.85%, and the reflectance to the optical path 02 may be set to about 0.15%.

Whereas if an optical member is added on the optical path 02 to set a receiving light amount of the reference light, an optical filter of which transmittance is determined in advance is inserted between the beam splitting mirror 126 and the mirror 127, or between the mirror 127 and the beam splitting mirror 128. For example, if a ratio of the transmission and the reflection by the beam splitting mirror 126 is fifty-fifty, the measurement light will be 50*0.15%=0.075, thus, an optical filter with transmittance of 0.15% may be inserted so as to bring the reference light close to 0.075 of the measurement light.

Accordingly, the receiving light amount of the reference light can be set. Here, the transmittance of the optical filter to be inserted is determined, based on the attenuation rate of the measurement light, so as to make the light amounts of the measurement light and the reference light are similar to each other.

When the receiving light amount of the reference light is set based on the reflectance and the transmittance of the beam splitting mirrors 126, and 128, and the mirror 127 included in the optical path 02, the transmittance of the beam splitting mirror 126 and the reflectance of the beam splitting mirror 128 and the mirror 127 are changed. Accordingly, the receiving light amount of the reference light can be set. Here, the transmittance and the reflectance of the mirror are determined, based on the attenuation rate of the measurement light, so as to make the light amounts of the measurement light and the reference light are similar to each other.

### (Switching of Laser Irradiation and Adjustment of Light Amount of Light Source)

Switching of irradiation of the laser beam to the subject's eye by a shutter 108 is performed as follows. The shutter 108 switches a state in which incident light from the light source to the subject's eye is controlled and a state in which incident light from the light source to the subject's eye is not controlled First, in a state immediately after the start of measurement, the shutter 108 is in a light blocking state, not to irradiate the subject's eye with the laser beam.

Since the optical path 01 is blocked by the shutter 108, the measurement light does not return to the optical path 03. On the other hand, the reference light passes through the lens 102 and the diaphragm 103 from the light source 101, is reflected by the beam splitting mirror 126 to enter the optical path 02, is reflected by the mirror 127 and the beam splitting mirror 128, and is focused around the center of the effective pixel surface 301 of the image sensor 112 which is an area not used by the ring image thereon.

A central processing unit (CPU) 113 compares an output value of the spot image formed around the center and a predetermined value which is set in advance. Then, if the output value is equal to or less than the predetermined value (a case within an allowable range), the CPU 113 controls a drive circuit (not illustrated) of the shutter 108 to open the optical path 01. In other words, the state is switched to a state in which the subject's eye is irradiated with the laser beam. The predetermined value is, for example, a maximum value which does not have a harmful effect on the subject's eye. On the other hand, if the output value exceeds the predetermined value (a case out of the allowable range), the CPU 113 maintains the state that the optical path 01 is blocked, and controls the light amount of the laser light source not to exceed the predetermined value.

A state before the measurement in which the subject's eye is irradiated with the laser beam is described above. However, in a state during the measurement in which the subject's eye is irradiated with the laser beam, the CPU 113 compares the output value and the predetermined value. If the output value exceeds the predetermined value (a case out of the allowable range), the CPU 113 immediately controls the drive circuit (not illustrated) of the shutter 108 to close the optical path 01, and adjusts the light amount of the laser light source. The adjustment of the light amount of the laser light source by the CPU 113 serving as a light amount control unit is performed by controlling a current or a voltage of the laser light source. Accordingly, the light amount of the laser light source is reduced, and the output value becomes equal to or less than the predetermined value (within the allowable range).

Then, if the output value becomes equal to or less than the predetermined value (within the allowable range), the CPU 113 controls the drive circuit (not illustrated) of the shutter 108 to open the optical path 01, so that the subject's eye can be irradiated with the laser beam.

However, if the output value does not become equal to or less than the predetermined value (within the allowable range) by controlling the light amount of the laser light source, the CPU 113 causes the monitor 200 to display a warning or the like, because of the possibility of apparatus failure. It the warning is displayed, an examiner can surely recognize the abnormality.

### (Flowcharts)

### (Flowchart for Entire Apparatus)

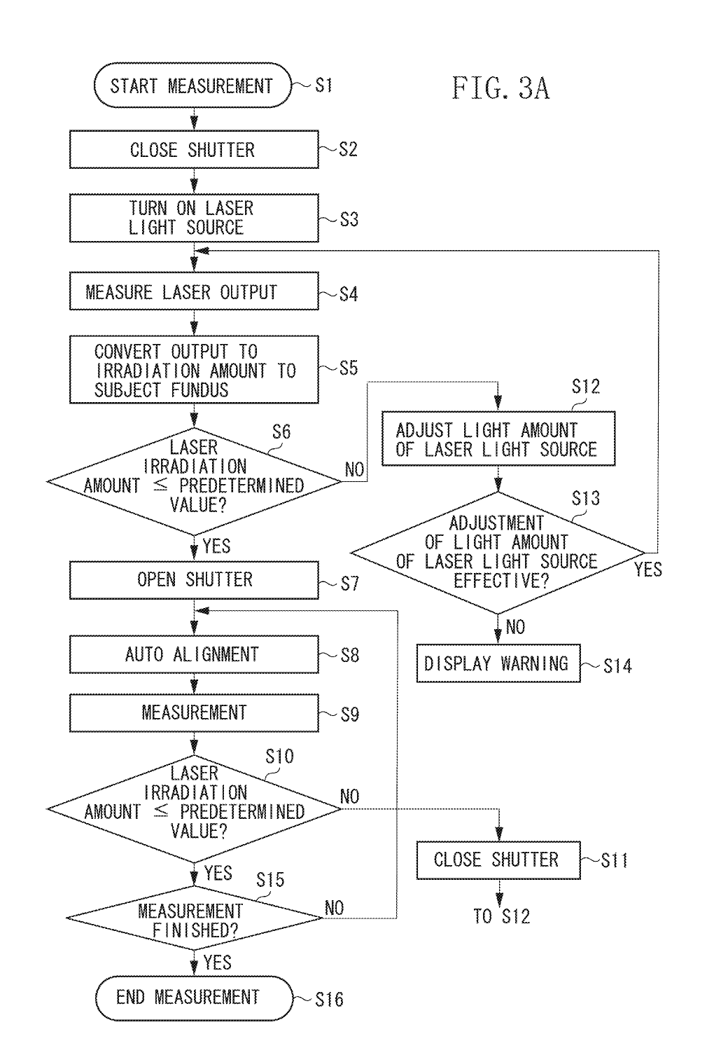

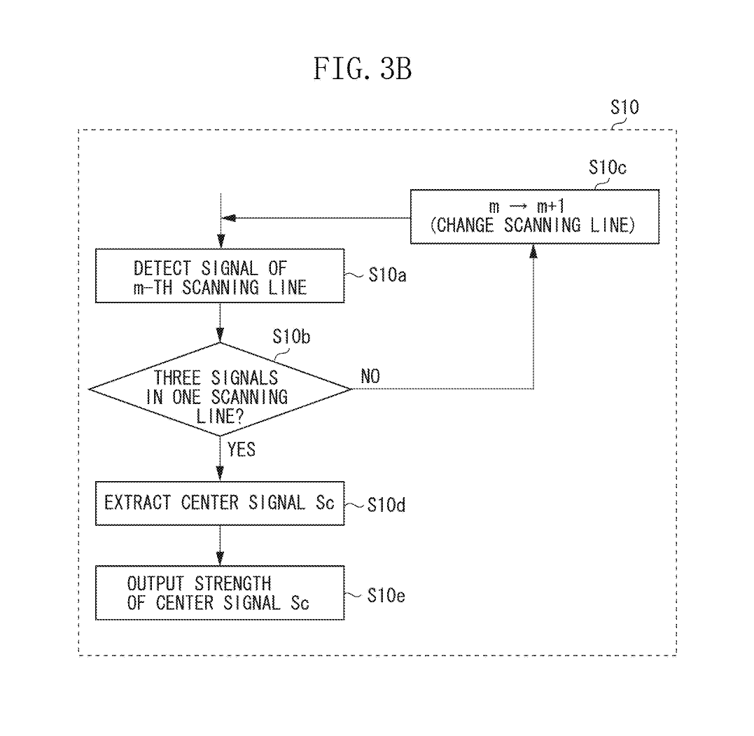

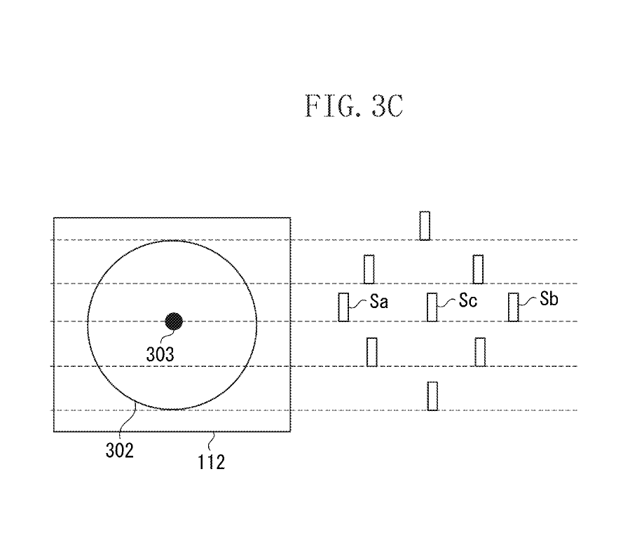

The above-described configuration is described below based on a flowchart illustrated in FIG. 3A together with the block diagram illustrated in FIG. 1B. The CPU 113 in FIG. 1B entirely controls the laser light source 101, the shutter 108, the monitor 200, and so on.

In step S1 in FIG. 3A, when the measurement is started, and in step S2 in FIG. 3A, the laser light is blocked, and the CPU 113 confirms that the laser light is not emitted outside of the apparatus. Then, the operation proceeds to processing, in step S3 in FIG. 3A, for generating the laser light, and the laser light source 101 (also serving as the measurement light source) is turned on. In the processing, in step S4 in FIG. 3A, for measuring an output of the laser light, the image sensor 112 serving as an output measurement unit measures the laser output.

Next, in conversion processing, in step S5 in FIG. 3A, based on the laser output and a conversion formula, which is stored in a storage unit (not illustrated), for converting a laser light output into an irradiation amount with which a fundus of a subject's eye is irradiated, the output is converted into the irradiation amount with which the fundus of the subject's eye is irradiated. In other words, a light amount incident on the subject's eye is determined.

In step S6 in FIG. 3A, based on the determination of proportion of the converted irradiation amount, if the converted irradiation amount is equal to or less than a predetermined value (YES in step S6), then in step S7 in FIG. 3A, the CPU 113 switch the shutter 108 from close to open. In step S8 in FIG. 3A, the above-described auto alignment is performed, and in step S9 in FIG. 3A, the measurement with use of the laser light is performed.

In other words, an irradiation step for irradiating the subject's eye by the irradiation optical system provided on the optical path between the light source and the subject's eye, and a first image forming step for forming an image of the light source reflected by the subject's eye as a first image by the first image forming optical system including the first optical path splitting member for splitting the optical path in the irradiation optical system are started. On the other hand, regarding the measurement of the laser output (from step S4 to step S6, and from step S9 to step S10), there is a second image forming step for forming an image of the light source without through the subject's eye as a second image on a different position from the first image by the second image forming optical system including the second optical path splitting member for splitting the optical path in the irradiation optical system.

If the converted irradiation amount is larger than the predetermined value (NO in step S6), then in step S12 in FIG. 3A, the CPU 113 serving as a light amount adjustment unit performs light amount adjustment processing to adjust the laser light amount. In the processing for adjusting the light amount in step S13, the CPU 113 determines whether the adjustment is effective or not by checking whether a value of the laser light amount has been changed. If the light amount adjustment is effective (YES in step S13), the operation returns to step S4 in FIG. 3A.

If the light amount adjustment is not effective (NO in step S13), then in step S14 in FIG. 3A, the CPU 113 cause the monitor 200 serving as a unit for informing an uncontrollable state to display a warning in a visual way or sound a warning in an auditory way. A state for giving a warning corresponds to a state in which an abnormality or a failure occurs in the apparatus, for example, a state in which an output of the laser light source cannot be controlled, a state in which an abnormal output is detected because of breakage of the optical member, or the like.

When the measurement with use of the laser light is performed in step S9 in FIG. 3A, confirmation of the laser output and conversion of the laser irradiation amount can be simultaneously performed, thus in step S10 in FIG. 3A, the CPU 113 determines whether the laser irradiation amount is equal to or less than the predetermined value. If the laser irradiation amount is equal to or less than the predetermined value (YES in step S10), the operation proceeds to step S15, and the CPU 113 determines whether an appropriate measurement value is obtained and the measurement can be finished. If it is determined that the appropriate measurement value is obtained (YES in step S15), then in step S16, the CPU 113 ends the measurement. In step S10 in FIG. 3A, if the laser irradiation amount is not equal to or less than the predetermined value (NO in step S10), in step S11, the CPU 113 closes the shutter 108 to block the optical path 01, and then in step S12, the CPU 113 adjusts the light amount of the laser light source. In step S15 in FIG. 3A, if the appropriate measurement value is not obtained and the measurement cannot be finished (NO in step S15), the operation is returned to step S8 and the CPU 113 performs position adjustment and measurement again.

Here, the confirmation of the laser output and the conversion of the laser irradiation amount can be simultaneously performed when the measurement with use of the laser light is performed, thus the processing thereof is described with reference to a flowchart in FIG. 3B, which illustrates the operations in step S10 in FIG. 3A in details, together with signal detection illustrated in FIG. 3C. Instep S10a, an optical image in the image sensor 112 is sequentially detected by each scanning line. At that time, in step S10b, it is determined whether one scanning line includes three signals. If the scanning line includes three signals (YES in step S10b), then in step S10d, a center signal Sc is extracted. In step S10e, an intensity of the center signal Sc is output. If the scanning line does not include three signals (NO in step S10b), then in step S10c, a position is changed from that of the m-th scanning line to that of the (m+l)-th scanning line, and the operation is repeated until it is determined that the scanning line includes three signals.

As described above, according to the present exemplary embodiment, an output of a reference light is monitored during the measurement, and the measurement can be performed while confirming that the output value is within the allowable range. Further, according to the present exemplary embodiment, if the output value is an abnormal value (not within the allowable range), the optical path is blocked, and the light amount of the laser light source can be adjusted to be reduced. Therefore, it can be assured that a subject's eye is irradiated with an appropriate laser beam.

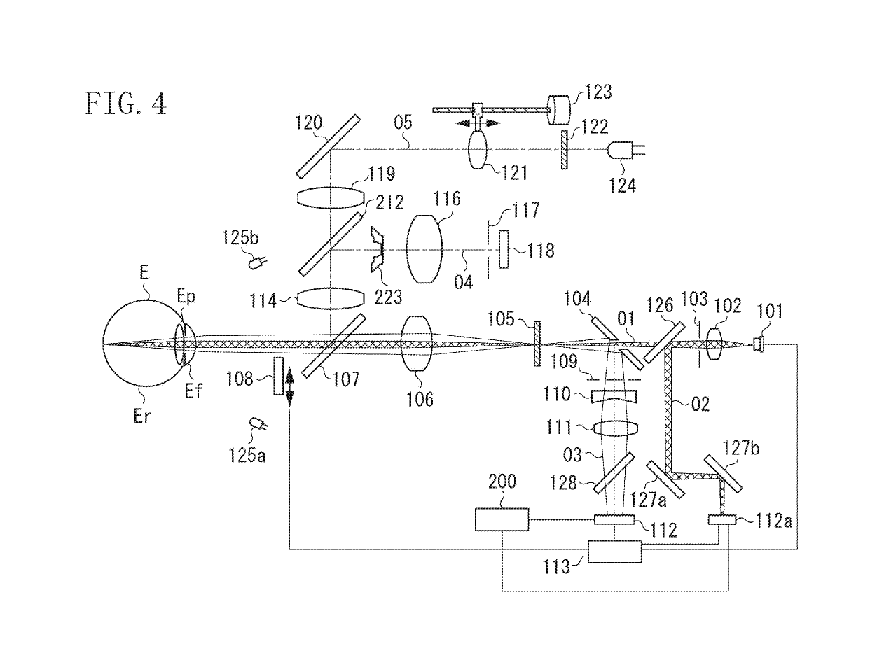

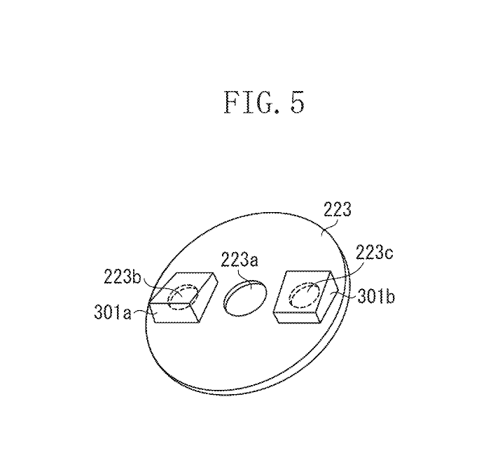

According to the above-described exemplary embodiment, the same image sensor 112 is used for both capturing of a measurement ring image and capturing of a reference spot image, and in a normal situation, a measurement spot image is supposed to overlap with a reference spot image, however, the measurement ring image and the reference spot image are relatively displaced so as not to overlap each other by the prism 110 or the like. However, the present invention is not limited to this configuration. As illustrated in FIG. 4, it can be configured that a measurement ring image and a reference spot image is separately captured by different image sensors 112 and 112a so that a position of a measurement ring image may not overlap with a position of a reference spot image. In FIG. 4, a reference spot image is formed on the image sensor 112a by a light beam reflected by the beam splitting mirror 126, and mirrors 127a and 127b.

According to the above-described exemplary embodiment, an image of the light source reflected by a subject's eye (i.e., a first image), which is supposed to be formed on the center position of the image sensor in a normal situation, is formed as a ring image displaced in each meridian direction by an action of the prism 110 so as not to overlap with a position of a reference spot image. However, the present invention is not limited to this configuration. For example, a displacement member may be provided on the reference optical path so that an image of the light source reflected by a cornea of a subject's eye (i.e., a first image) is formed on the center position of the image sensor, whereas an image of the light source without through the subject's eye is formed as a second image on a position deviated from the center position of the same image sensor.

More specifically, a reference spot image can be formed on a position deviated from the center position of the image sensor 112 by rotating and displacing the mirror 127.

According to the above-described exemplary embodiment, if an output of a reference light exceed the allowable range, it is described that the optical path in the irradiation optical system for irradiating a subject's eye is switched to a blocked state by the shutter 108, however, the present invention is not limited to this configuration. In other words, in a case where the light amount adjustment of the light source is not performed, the light source maybe switched to a light-off state without using the shutter 108.

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™, a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-234649 filed Oct. 24, 2012, which is hereby incorporated by reference herein in its entirety.

What is claimed is:

1. An ophthalmologic apparatus comprising:
a light source configured to irradiate a subject's eye;
an irradiation optical system arranged between the light source and the subject's eye;
a first image forming optical system configured to include a first optical path splitting member for splitting an optical path of the irradiation optical system and form an image of a return light from the subject's eye as a first image; and
a second image forming optical system configured to include a second optical path splitting member for splitting an optical path of the irradiation optical system and form an image of a light output from the light source without through the subject's eye as a second image on a different position from that of the first image.

2. The ophthalmologic apparatus according to claim 1, wherein the first image and the second image are formed on a same image sensor.

3. The ophthalmologic apparatus according to claim 2, wherein the first image is a ring image, and the second image is a spot image.

4. The ophthalmologic apparatus according to claim 3, wherein the spot image is formed inside the ring image.

5. The ophthalmologic apparatus according to claim 1 further comprising a switching unit configured to, in a case where an output of the second image exceeds an allowable range, switch a state in which a light from the light source incident on the subject's eye is controlled and a light-off state of the light source.

6. The ophthalmologic apparatus according to claim 1 further comprising a light amount control unit configured to, in a case where an output of the second image exceeds an allowable range, control a light amount of the light source to be reduced based on the output of the second image.

7. The ophthalmologic apparatus according to claim 2, wherein a reflectance or a transmittance of an optical member configuring the first image forming optical system or the second image forming optical system is set so that receiving light amounts of the image sensor are equalized with respect to the first image and the second image.

8. A method for ophthalmological control, the method comprising:
irradiating a subject's eye by an irradiation optical system arranged between a light source and the subject's eye;
forming an image of the light source reflected by the subject's eye as a first image by a first image forming optical system including a first optical path splitting member for splitting an optical path of the irradiation optical system; and
forming an image of the light source without through the subject's eye as a second image on a different position from that of the first image by a second image forming optical system including a second optical path splitting member for splitting an optical path of the irradiation optical system.

9. A non-transitory storage medium storing an ophthalmological control program for causing a computer to execute each step in a method according to claim 8.
